(19) United States
(12) Patent Application Publication
(10) Pub. No.: US 2005/0244443 A1
(43) Pub. Date: Nov. 3, 2005

(54) Cosmetic concentrate

(75) Inventors: Sabine Stiller, Hamburg (DE); Jens Schulz, Schenefeld (DE)

Correspondence Address: GREENBLUM & BERNSTEIN, P.L.C., 1950 ROLAND CLARKE PLACE, RESTON, VA, 20191, US

(73) Assignee: BEIERSDORF AG, Hamburg (DE)

(21) Appl. No.: 11/133,295

(22) Filed: May 20, 2005

Related U.S. Application Data
(63) Continuation of application No. PCT/EP03/50845, filed on Nov. 18, 2003.

(30) Foreign Application Priority Data
Nov. 21, 2002 (DE) 102 54 334.8

(51) Int. Cl. A61K 7/021; A61K 6/00; A61K 7/00

(52) U.S. Cl. 424/401

(57) ABSTRACT

A process for preparing an O/W emulsion concentrate. The process comprises subjecting an O/W emulsion comprising 20% to 95% by weight of one or more lipids, 0.5% to 80% by weight of microfine amphiphilic metal oxide particles, amphiphilic polymer particles, and/or modified polysaccharide particles and water to spray-drying and/or freeze-drying.

### CROSS-REFERENCE TO RELATED APPLICATIONS

The present application is a continuation of International Application No. PCT/EP2003/050845, filed Nov. 18, 2003, the entire disclosure whereof is expressly incorporated by reference herein, which claims priority under 35 U.S.C. § 119 of German Patent Application No. 102 54 334.8, filed Nov. 21, 2002.

### BACKGROUND OF THE INVENTION

1. Field of the Invention

The present invention relates to a process for the preparation of an O/w emulsion, which is characterized in that an O/W emulsion comprising
a) a lipid phase in a concentration of from 20 to 95% by weight,
b) one or more solid carriers in the form of microfine solid particles from the classes of amphiphilic metal oxides, amphiphilic polymer particles or modified polysaccharides in a concentration of from 0.5 to 80% by weight, in each case based on the total weight of the preparation, and
c) an aqueous phase,
   besides optionally further cosmetic and/or dermatological active ingredients, auxiliaries and additives,
   is dried by spray-drying and/or freeze-drying.

2. Discussion of Background Information

The desire to appear beautiful and attractive is naturally rooted in man. Even if the beauty ideal has undergone changes over the course of time, the desire for a flawless outward appearance has always been the aim of humans. The condition and the appearance of the skin and of the skin appendages, i.e. the hair and of the nails, is a significant part of a beautiful and attractive outward appearance.

The skin is the largest human organ. Among its many functions (for example temperature regulation and as a sensory organ), the barrier function, which prevents the skin (and thus ultimately the entire organism) from drying out, is probably the most important. At the same time, the skin acts as a protective device against the penetration and the absorption of external substances and UV radiation. This barrier function is effected by the epidermis which, being the outermost layer, forms the actual protective sheath against the environment. Being about one tenth of the overall thickness, it is also the thinnest layer of the skin.

In order that the skin can completely fulfil its biological functions it requires regular cleansing and care. Cleansing the skin serves to remove dirt, perspiration and remains of dead skin particles which form an ideal breeding ground for pathogens and parasites of all types. Skin care products, generally creams, ointments or lotions, serve mostly to moisturize and regrease the skin. Active ingredients are often added to them which regenerate the skin and, for example, are intended to prevent and reduce its premature ageing (e.g. the appearance of wrinkles, lines).

Skin care products usually consist of emulsions. Emulsions are generally understood as meaning heterogeneous systems which consist of two liquids which are immiscible or only of limited miscibility and which are usually referred to as phases and in which one of the two liquids is dispersed in the form of very fine droplets in the other liquid. Outwardly and viewed with the naked eye, emulsions appear homogeneous.

If the two liquids are water and oil and oil droplets are present in finely dispersed form in water, this is an oil-in-water emulsion (O/W emulsion, e.g. milk). The basic character of a O/W emulsion is defined by the water. In the case of a water-in-oil emulsion (W/O emulsion, e.g. butter), the principle is reversed, the basic character here being determined by the oil.

The trend away from genteel pallor toward “healthy, sporty brown skin” has been unbroken for years. In order to attain this, people expose their skin to solar radiation since this, brings about pigment formation in the sense of melanine formation. However, the ultraviolet radiation of sunlight also has a harmful effect on the skin. Besides the acute damage (sunburn), long-term damage, such as suffering from an increased risk of skin cancer in cases of excessive irradiation with light from the UVB region (wavelength: 280-320 nm), arises. Moreover, the excessive effect of UVB and UVA radiation (wavelength: 320-400 nm) leads to a weakening of the elastic and collagenous fibres of connective tissue. This leads to numerous phototoxic and photoallergic reactions and results in premature skin ageing.

To protect the skin, a number of photoprotective filters have therefore been developed which can be used in cosmetic preparations. These UVA and UVB filters are summarized in most industrialized countries in the form of positive lists such as Annex 7 of the Cosmetics Directive.

Due to their water content, conventional cosmetic and/or dermatological emulsions, for example sunscreen creams or lotions, have a number of disadvantages:
They have a high weight, which leads to higher energy consumption and higher costs during transportation.
They have a relatively large volume, which leads to lower transport capacities during transportation.
Particularly low viscosity emulsions are significantly more difficult to store and transport since the preparations can “leak”.
The application concentrations of the ingredients (e.g., UV filter concentration and thus UV filter performance) are already predefined for the consumer. Individual adaptation to the conditions at the site of use is no longer possible.

It would be desirable to overcome the disadvantages of the prior art and have available a “dry” emulsion and a process for preparing the same.

### SUMMARY OF THE INVENTION

The present invention provides a process for preparing an O/W emulsion concentrate. This process comprises subjecting an O/W emulsion comprising
(a) 20% to 95% by weight of one or more lipids;
(b) 0.5% to 80% by weight of microfine solid particles selected from amphiphilic metal oxide particles, amphiphilic polymer particles, modified polysaccharide particles and combinations thereof; and
(c) water;
   to spray-drying and/or freeze-drying.

In one aspect of the process, the starting O/W emulsion may comprise at least 30% by weight of component (a), e.g., at least 50% by weight thereof.

In another aspect of the process, the O/W emulsion may comprise from 1% to 50% by weight of component (b), e.g., at least 3% by weight and/or not more than 40% by weight thereof.

In yet another aspect of the process the spray-drying and/or freeze-drying may be conducted to a water content of less than 8% by weight, e.g., to a water content of less than 5% by weight.

In a still further aspect, the microfine solid particles may have an average particle size of less than 100 μm, e.g., less than 50 μm.

In another aspect, component (b) may comprise metal oxide particles. For example, the metal oxide may comprise one or more of titanium dioxide, zinc oxide and iron oxides.

In another aspect, the microfine solid particles may comprise at least one of boron nitride, talc and latex.

In yet another aspect of the process, component (b) may comprise polymer particles, for example, polymer particles comprising one or more polymers selected from polycarbonates, polyethers, polyethylene, polypropylene, polyvinyl chloride, polystyrene, polyamides, and polyacrylates.

In a still further aspect, component (b) may comprise modified polysaccharide particles, for example, particles comprising a starch ether and/or a starch ester. Preferably, the modified polysaccharide particles have an average particle diameter of less than 20 μm.

In another aspect of the process, the O/W emulsion may further comprise one or more polyols. Preferably, the one or more polyols are present in a concentration of from 5% to 40% by weight.

The present invention also provides a process for preparing an O/w emulsion concentrate, comprising subjecting an O/W emulsion that comprises
(a) 50% to 95% by weight of one or more lipids;
(b) 3% to 40% by weight of microfine solid particles having an average particle size of less than 50 μm and being selected from amphiphilic particles of at least one of titanium dioxide, zinc oxide and iron oxides; amphiphilic particles of at least one of a polycarbonate, a polyether, polyethylene, polypropylene, polyvinyl chloride, polystyrene, a polyamide, and a polyacrylate; particles of a starch ether and/or a starch ester; and combinations thereof; and
(c) water;
   to spray-drying and/or freeze-drying to obtain an O/W emulsion concentrate having a water content of less than 5% by weight.

The present invention also provides a process as set forth above, including the various aspects thereof, which further comprises mixing the O/W emulsion concentrate with one or more substances selected from water-soluble substances, readily volatile substances, auxiliaries and additives and/or further comprises mixing the O/W emulsion concentrate with one or more substances selected from granulating agents and tablet auxiliaries to obtain a second mixture. For example, the O/W emulsion concentrate may be mixed with at least one substance selected from carbonates, bicarbonates and acids that are solid at room temperature. The process may further comprise granulating the resultant mixture and/or pressing the resultant mixture into a tablet.

The present invention also provides a process for preparing an O/W emulsion, which process comprises combining the O/W emulsion concentrate that is obtainable by the process set forth above, including the various aspects thereof, with water.

The present invention also provides the products that are obtainable by the processes set forth above, including the various aspects thereof.

The present invention also provides a cosmetic preparation which comprises the concentrate obtainable by the process set forth above. In one aspect, this preparation may further comprise one or more UV photoprotective filter substances.

The present invention further comprises a composition for the preparation of an O/W emulsion. The composition comprises a mixture of (a) one or more lipids, (b) microfine solid particles selected from amphiphilic metal oxide particles, amphiphilic polymer particles, modified polysaccharide particles and combinations thereof, and (c) at least one substance selected from carbonates, bicarbonates, and acids that are solid at room temperature such as, e.g., one or more of sodium bicarbonate, potassium bicarbonate and citric acid.

According to the present invention, it is preferred for the lipid phase to be present in a concentration of from 30 to 95% by weight and particularly preferably in a concentration of from 50 to 95% by weight, in each case based on the total weight of the preparation, in the emulsion according to the invention.

According to the invention, it is further preferred if one or more solid carriers in the form of microfine solid particles are present in a concentration of from 1 to 50% by weight and particularly preferably in a concentration of from 3 to 40% by weight, in each case based on the total weight of the emulsion.

According to the invention, it is preferred if the O/W emulsion according to the invention has, after drying, a water content of less than 8% by weight and particularly preferably less than 5% by weight, in each case based on the total weight of the emulsion concentrate.

It is particularly surprising that the dry emulsions of the present invention can be “reemulsified” by simply adding water, i.e. that an O/W emulsion reforms, which is usually not the case for “dried” emulsions.

Although Y. Kawashima et al [Int. J. Pharmazeut., 86 (1992) 25-33, Drug Development and Industrial Pharmacy, 18(9), 919-937 (1992) and Chem. Pharm. Bull. 39(6) 1528-1531 (1991)] and H. G. Kristensen et al. [Euro. J. Pharmaceutics and Biopharmaceutics 53 (2002) 147-153 and Int. J. Pharmaceutics 212 (2001) 187-194, 195-202] describe dried, redispersible emulsions, these emulsions are stabilized with the help of hydroxypropylmethylcellulose. The emulsions described therein also have a structure which is nowhere nearly as complex as cosmetic and/or dermatological emulsions. For this reason, these publications could in no way point to the present invention. Compared with the redispersible emulsions known to date, the emulsions according to the invention have a particularly pleasant feel on the skin and can be combined with a large number of cosmetic active ingredients, auxiliaries and additives.

The oil phase of the emulsion according to the invention, i.e., the lipophilic organic constituents, are advantageously chosen from polar oils, for example from lecithins and fatty acid triglycerides, namely the triglycerol esters of saturated and/or unsaturated, branched and/or unbranched alkanecarboxylic acids having a chain length of from 8 to 24, in particular 12 to 18, carbon atoms. The fatty acid triglycerides can, for example, be chosen advantageously from synthetic, semisynthetic and natural oils, such as, for example, cocoglyceride, olive oil, sunflower oil, soya oil, peanut oil, rapeseed oil, almond oil, palm oil, coconut oil, castor oil, wheatgerm oil, grapeseed oil, thistle oil, evening primrose oil, macadamia nut oil and the like.

Also advantageous according to the invention are, for example, natural waxes of animal and vegetable origin, such as, for example, beeswax and other insect waxes, and berry wax, shea butter and/or lanolin (wool wax).

For the purposes of the present invention, further advantageous polar oil components can also be chosen from the esters of saturated and/or unsaturated, branched and/or unbranched alkanecarboxylic acids having a chain length of from 3 to 30 carbon atoms and saturated and/or unsaturated, branched and/or unbranched alcohols having a chain length of from 3 to 30 carbon atoms, and from the esters of aromatic carboxylic acids and saturated and/or unsaturated, branched and/or unbranched alcohols having a chain length of from 3 to 30 carbon atoms. Such ester oils can be chosen advantageously from octyl palmitate, octyl cocoate, octyl isostearate, octyldodeceyl myristate, octyldodecanol, cetearyl isononanoate, isopropyl myristate, isopropyl palmitate, isopropyl stearate, isopropyl-oleate, n-butyl stearate, n-hexyl laurate, n-decyl oleate, isooctyl stearate, isononyl stearate, isononyl isononanoate, 2-ethylhexyl palmitate, 2-ethylhexyllaurate, 2-hexyldecyl stearate, 2-octyldodecyl palmitate, stearyl heptanoate, oleyl oleate, oleyl erucate, erucyl oleate, erucyl erucate, tridecyl stearate, tridecyl trimellitate, and synthetic, semisynthetic and natural mixtures of such esters, such as, for example, jojoba oil.

In addition, the oil phase can be chosen advantageously from dialkyl ethers and dialkyl carbonates, advantageous examples thereof being dicaprylyl ether (Cetiol OE) and/or dicaprylyl carbonate, which are available, for example, under the trade name Cetiol CC from Cognis.

It is also preferred to choose the oil component or the oil components from isoeicosane, neopentyl glycol diheptanoate, propylene glycol dicaprylate/dicaprate, caprylic/capric/diglyceryl succinate, butylene glycol dicaprylate/dicaprate, cocoglycerides (e.g. Myritol® 331 from Henkel), C₁₂₋₁₃-alkyl lactate, di-C₁₂₋₁₃-alkyl tartrate, triisostearin, dipentaerythrityl hexacaprylate/hexacaprate, propylene glycol monoisostearate, tricaprylin, and dimethyl isosorbide. It is particularly advantageous if the oil phase of the formulations according to the invention has a content of C₁₂₋₁₅-alkyl benzoate, or consists entirely thereof.

Advantageous oil components are also, for example, butyloctyl salicylate (for example that available under the trade name Hallbrite BHB from CP Hall), hexadecyl benzoate and butyloctyl benzoate and mixtures thereof (Hallstar AB) and/or diethylhexyl naphthalate (Corapan®TQ from Haarmann & Reimer).

Any mixtures of such oil and wax components can also be used advantageously for the purposes of the present invention.

According to the invention, the lipid phase can comprise the polar oil components in a concentration of up to 80% by weight, based on the total weight of the lipid phase. The weight data refer to the composition of the preparation before drying by means of freeze-drying and/or spray-drying.

In addition, the oil phase can likewise advantageously also comprise nonpolar oils, for example those which are chosen from the group of branched and unbranched hydrocarbons and hydrocarbon waxes, in particular mineral oil, vaseline (petrolatum), paraffin oil, squalane and squalene, polyolefins, hydrogenated polyisobutenes and isohexadecane. Among the polyolefins, polydecenes and hydrogenated polyisobutenes are the preferred substances.

The nonpolar oil components can advantageously be present in the emulsions according to the invention in a concentration of up to 80% by weight, based on the total weight of the lipid phase. The weight data refers to the composition of the preparation before drying by means of freeze-drying and/or spray-drying.

The oil phase can also advantageously have a content of cyclic or linear silicone oils, or consist entirely of such oils, although it is preferred to use an additional content of other oil phase components apart from the silicone oil or the silicone oils.

Silicone oils are high molecular weight synthetic polymeric compounds in which silicon atoms are joined in a catenated and/or reticular manner via oxygen atoms and the remaining valencies of the silicon are saturated by hydrocarbon radicals (in most cases methyl groups, more rarely ethyl, propyl, phenyl groups etc.). Systematically, the silicone oils are referred to as polyorganosiloxanes. The methyl-substituted polyorganosiloxanes, which represent the most important compounds of this group in terms of amount and are characterized by the following structural formula 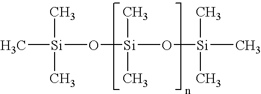
are also referred to as polydimethylsiloxane or dimethicone (INCI). Dimethicones come in various chain lengths and with various molecular weights.

For the purposes of the present invention, particularly advantageous polyorganosiloxanes are, for example, dimethylpolysiloxanes [poly(dimethylsiloxane)], which are available, for example, under the trade name Abil 10 to 10 000 from Th. Goldschmidt. Also advantageous are phenylmethylpolysiloxanes (INCI: Phenyl Dimethicone, Phenyl Trimethicone), cyclic silicones (octamethylcyclotetrasiloxane and decamethylcyclopentasiloxane), which are also referred to in accordance with INCI as Cyclomethicone, amino-modified silicones (INCI: Amodimethicones) and silicone waxes, e.g. polysiloxane-polyalkylene copolymers (INCI: Stearyl Dimethicone and Cetyl Dimethicone) and dialkoxydimethylpolysiloxane (Stearoxy Dimethicone and Behenoxy Stearyl Dimethicone), which are available as various Abil-wax grades from Th. Goldschmidt. However, other silicone oils are also to be used advantageously for the purposes of the present invention, for example cetyldimethicone, hexamethylcyclotrisiloxane, polydimethylsiloxane, poly(methylphenylsiloxane).

According to the invention, particularly preferred silicones are dimethicone and cyclomethicone.

The fraction of silicone oil in the lipid phase can advantageously be from 20 to 100% by weight and particularly preferably from 30 to 60% by weight, in each case based on the total weight of the lipid phase.

The stabilizing solid carriers in the form of microfine solid particles can be treated superficially to repel water (“coated”), the intention being to form and/or retain an amphiphilic character of these solid particles. The surface treatment can consist in providing the solid particles with a thin hydrophobic or hydrophilic layer by processes known per se.

The average particle diameter of the microfine solid carriers (solid particles) used as stabilizer is preferably chosen to be less than 100 μm, particularly advantageously less than 50 μm. Here, it is essentially unimportant in what form (platelets, rods, beads etc.) or modification the solid particles used are present.

Preferably, the microfine solid carriers are chosen from the group of amphiphilic metal oxide pigments. Of particular advantage are:
titanium dioxides (coated and uncoated): e.g. Eusolex T-2000 from Merck, titanium dioxide MT-100 Z from Tayca Corporation
zinc oxides, e.g. Z-Cote and Z-Cote HP1 from BASF AG, zinc oxide NDM from H&R and MZ-505M and MZ-303M from Tayca Corporation
iron oxides

Furthermore, it is advantageous if the (sub)microfine solid carriers are chosen from the following group: boron nitride, talc, latex particles.

Further advantageous microfine solid particles are chosen from the group of polymer particles.

For the purposes of the present invention, advantageous microfine polymer particles are, for example, those of polycarbonates, polyethers, polyethylene, polypropylene, polyvinyl chloride, polystyrene, polyamides, polyacrylates and the like.

According to the invention, for example, microfine polyamide particles which are available under the trade name SP-500 from TORAY are advantageous. Also advantageous are polyamide 6 (also: nylon 6) particles and polyamide 12 (also: nylon 12) particles. Polyamide 6 is the polyamide constructed from ε-aminocaproic acid (6-aminohexanoic acid) or ε-caprolactam [poly(ε-caprolactam)], and polyamide 12 is a poly(ε-laurolactam) of ε-laurolactam. Of advantage for the purposes of the present invention are, for example, Orgasol® 1002 (polyamide 6) and Orgasol® 2002 (polyamide 12) from ELF ATOCHEM. Further advantageous polymer particles are microfine polymethacrylates. Such particles are available, for example, under the trade name POLYTRAP® from DOW CHEMICAL.

It is particularly advantageous, although not mandatory, for the used microfine polymer particles to be coated superficially. This surface coating can consist in providing the polymer particles with a thin hydrophilic layer by processes known per se. Advantageous coatings consist, for example, of TiO₂, ZrO₂ or else further polymers, such as, for example, polymethyl methacrylate.

Particularly advantageous microfine polymer particles for the purposes of the present invention are also obtainable by the process described in the U.S. Pat. No. 4,898,913 for the hydrophilic coating of hydrophobic polymer particles.

Furthermore, it is advantageous to choose microfine solid particles from the group of modified, water-dispersible, nonswelling polysaccharides. Modified polysaccharides for the purposes of the present invention are obtainable, for example, by reacting starch with mono-, bi- or polyfunctional reagents or oxidizing agents in reactions which proceed in a largely polymer-like manner. Such reactions are essentially based on modifications of the hydroxyl groups of the polyglucans by etherification, esterification or selective oxidation. This produces, for example, so-called starch ethers and starch esters of the structural formula (I) 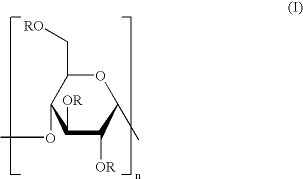
in which R can, for example, be a hydrogen and/or an alkyl and/or aralkyl radical (in the case of the starch ethers) or a hydrogen and/or an organic and/or inorganic acid radical (in the case of the starch esters). Starch ethers and starch esters are advantageous modified polysaccharides for the purposes of the present invention.

Particularly advantageous starch ethers are, for example, those obtainable by etherification of starch with tetramethylol acetylenediurea and which are referred to as Amylum non mucilaginosum (nonswelling starch).

Also particularly advantageous are starch esters and salts thereof, for example the sodium and/or aluminium salts of half-esters of starch with low degrees of substitution, in particular sodium starch n-octenylsuccinate in which R has the following structure 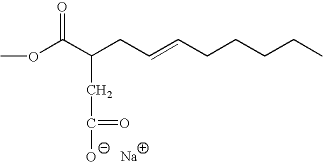
and which is available, for example, under the trade name Amiogum® 23 from CERESTAR, and aluminium starch octenylsuccinates, in particular those available under the trade names Dry Flo® Elite LL and Dry Flo® PC from CERESTAR.

It is advantageous to choose the average particle diameter of the modified polysaccharides used to be less than 20 μm, particularly advantageously less than 15 μm. The combination of one or more solids of different chemical classes with one another is also in accordance with the invention.

It is also in accordance with the invention if an O/W emulsion according to the present invention comprises water-soluble cosmetic and/or dermatological active ingredients, auxiliaries and/or additives.

The process for the preparation of such an O/W emulsion according to the invention, wherein an O/W emulsion according to the invention which has been dried by spray-drying or freeze-drying is mixed in a mixing device with water-soluble and/or readily volatile active ingredients, auxiliaries and/or additives, also forms a part of the present invention.

“Readily volatile” according to the invention means that these compounds have a boiling point of at most 30° C. An O/W emulsion which is prepared by such a process also forms a part of the present invention.

According to the invention, the emulsion according to the invention can comprise water-soluble and/or water-dispersible ingredients. These are advantageously added to the preparation according to the invention after the freeze-drying or spray-drying.

Water-soluble or water-dispersible ingredients according to the invention are, for example, alcohols, diols or polyols of low carbon number, and ethers thereof, preferably ethanol, isopropanol, propylene glycol, ethylene glycol, ethylene glycol monoethyl or monobutyl ether, propylene glycol monomethyl, monoethyl or monobutyl ether, diethylene glycol monomethyl or monoethyl ether and analogous products.

The emulsions may also comprise one or more polyols chosen from sorbitol, propylene glycol and butylene glycol.

The polyols particularly preferred according to the invention are sorbitol and mannitol.

For the purposes of the present invention, it is advantageous if the cosmetic and/or dermatological emulsion according to the invention comprises a total amount of polyols of from 5.0 to 40.0% by weight, preferably from 7.5 to 35.0% by weight and very particularly preferably from 10.0 to 25.0% by weight, in each case based on the total weight of the preparation. The weight data refer here to the composition of the preparation before drying by means of freeze-drying and/or spray-drying.

Preparations for the purposes of the present invention preferably comprise at least one UV-A and/or UV-B filter substance. The formulations may, although not necessarily, optionally also comprise one or more organic and/or inorganic pigments as UV filter substances, which may be oil- or water-soluble.

Preferred inorganic pigments are metal oxides and/or other metal compounds which are insoluble or sparingly soluble in water, in particular oxides of titanium (TiO₂), zinc (ZnO), iron (e.g. Fe₂O₃), zirconium (ZrO₂), silicon (SiO₂), manganese (e.g. MnO), aluminium (Al₂O₃), cerium (e.g. Ce₂O₃), mixed oxides of the corresponding metals, and mixtures of such oxides, and barium sulphate (BaSO₄).

The titanium dioxide pigment may be present either in the rutile or anatase crystal modification and, for the purposes of the present invention, may advantageously be surface-treated (“coated”), the intention being, for example, to form and/or retain a hydrophilic, amphiphilic or hydrophobic character. This surface treatment can consist in providing the pigments with a thin hydrophilic and/or hydrophobic inorganic and/or organic layer by processes known per se. For the purposes of the present invention, the different surface coatings can also comprise water.

For the purposes of the present invention, described coated and uncoated titanium dioxides can also be used in the form of commercially obtainable oily or aqueous predispersions. Dispersion auxiliaries and/or solubilization promoters can advantageously be added to these predispersions.

The titanium dioxides according to the invention are characterized by a primary particle size between 10 nm to 150 nm.

| | | Additional | |
|---|---|---|---|
| | | constituents | |
| | | of the | |
| Trade name | Coating | predispersion | Manufacturer |
| MT-100TV | Aluminium hydroxide | — | Tayca |
| | Stearic acid | | Corporation |
| MT-100Z | Aluminium hydroxide | — | Tayca |
| | Stearic acid | | Corporation |
| MT-100F | Stearic acid | — | Tayca |
| | Iron oxide | | Corporation |
| MT-500SAS | Alumina, silica | — | Tayca |
| | Silicone | | Corporation |
| MT-100AQ | Silica | — | Tayca |
| | Aluminium hydroxide | | Corporation |
| | Alginic acid | | |
| Eusolex T-2000 | Alumina | — | Merck KgaA |
| | Simethicone | | |
| Eosolex TS | Alumina, stearic acid | — | Merck KgaA |
| Titanium dioxide | None | — | Degussa |
| P25 | | | |
| Titanium dioxide | Octyltrimethylsilane | — | Degussa |
| T805 | | | |
| (Uvinul TiO₂) | | | |
| UV-Titan X170 | Alumina | — | Kemira |
| | Dimethicone | | |
| UV-Titan X161 | Alumina, silica | — | Kemira |
| | Stearic acid | | |
| Tioveil AQ 10PG | Alumina | Water | Solaveil |
| | Silica | Propylene | Uniqema |
| | | glycol | |
| Mirasun TiW 60 | Alumina | Water | Rhone-Poulenc |
| | Silica | | |

For the purposes of the present invention, particularly preferred titanium dioxides are the MT-100 Z and MT-100 TV from Tayca Corporation, Eusolex T-2000 and Eusolex TS from Merck and the titanium dioxide T 805 from Degussa.

For the purposes of the present invention, zinc oxides can also be used in the form of commercially available oily or aqueous predispersions. Zinc oxide particles and predispersions of zinc oxide particles suitable according to the invention are characterized by a primary particle size of <300 nm and are available under the following trade names from the companies listed:

| Trade name | Coating | Manufacturer |
|---|---|---|
| Z-Cote HP1 | 2% Dimethicone | BASF |
| Z-Cote | / | BASF |
| ZnO NDM | 5% Dimethicone | H&R |
| MZ 707M | 7% Dimethicone | M. Tayca Corp. |
| Nanox 500 | / | Elementis |
| ZnO Neutral | / | H&R |

Particularly preferred zinc oxides for the purposes of the invention are Z-Cote HP1 from BASF and the zinc oxide NDM from Haarmann & Reimer.

The total amount of one or more inorganic pigments in the finished cosmetic preparation is advantageously from 0.1% by weight to 25% by weight, preferably from 0.5% by weight to 18% by weight.

An advantageous organic pigment for the purposes of the present invention is 2,2′-methylenebis(6-(2H-benzotriazol-2-yl)-4-(1,1,3,3-tetramethylbutyl)phenol) [INCI: Bisoctyltriazole], which is available under the tradename Tinosorb® M from CIBA-Chemikalien GmbH.

Advantageous UV-A filter substances for the purposes of the present invention are dibenzoylmethane derivatives, in particular 4-(tert-butyl)-4′-methoxydibenzoylmethane (CAS No. 70356-09-1), which is sold by Givaudan under the brand Parsol® 1789 and by Merck under the tradename Eusolex® 9020.

Further advantageous UV-A filter substances are phenylene-1,4-bis(2-benzimidazyl)-3,3′,5,5′-tetrasulphonic acid and its salts, particularly the corresponding sodium, potassium or triethanolammonium salts, in particular the phenylene-1,4-bis(2-benzimidazyl)-3,3′,5,5′-tetrasulphonic acid bis-sodium salt with the INCI name Bisimidazylate, which is available, for example, under the trade name Neo Heliopan AP from Haarmann & Reimer.

Also advantageous is 1,4-di(2-oxo-10-sulpho-3-bornylidenemethyl)benzene and salts thereof (particularly the corresponding 10-sulphato compounds, in particular the corresponding sodium, potassium or triethanolammonium salt), which is also referred to as benzene-1,4-di(2-oxo-3-bornylidenemethyl-10-sulphonic acid).

Further advantageous UV-A filter substances are hydroxybenzophenones, which are characterized by the following structural formula: 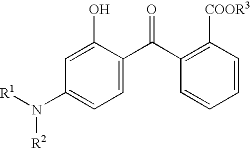
in which
R¹ and R², independently of one another, are hydrogen, C₁-C₂₀-alkyl, C₃-C₁₀-cycloalkyl or C₃-C₁₀-cycloalkenyl, where the substituents R¹ and R², together with the nitrogen atom to which they are bonded, can form a 5- or 6-membered ring and
R³ is a C₁-C₂₀-alkyl radical.

A particularly advantageous hydroxybenzophenone for the purposes of the present invention is hexyl 2-(4′-diethylamino-2′-hydroxybenzoyl)benzoate (also: aminobenzophenone), which is characterized by the following structure: 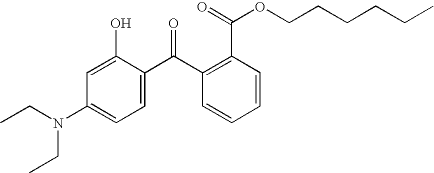
and is available under the trade name Uvinul A Plus from BASF.

Advantageous UV filter substances for the purposes of the present invention are also so-called broadband filters, i.e. filter substances which absorb both UV-A and also UV-B radiation.

Advantageous broadband filters or UV-B filter substances are, for example, bisresorcinyltriazine derivatives with the following structure: 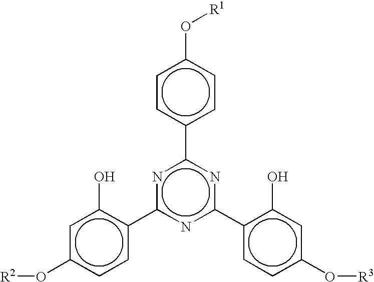
where R¹, R² and R³, independently of one another, are chosen from branched and unbranched alkyl groups having from 1 to 10 carbon atoms and hydrogen. Particular preference is given to 2,4-bis{[4-(2-ethylhexyloxy)-2-hydroxy]-phenyl}-6-(4-methoxyphenyl)-1,3,5-triazine (INCI: Aniso Triazine), which is available under the trade name Tinosorb® S from CIBA-Chemikalien GmbH.

Particularly advantageous preparations for the purposes of the present invention which are characterized by high or very high UV-A protection preferably comprise two or more UV-A and/or broadband filters, in particular dibenzoylmethane derivatives [for example 4-(tert-butyl)-4′-methoxydibenzoylmethane], benzotriazole derivatives [for example 2,2′-methylenebis(6-(2H-benzotriazol-2-yl)-4-(1,1,3,3-tetramethylbutyl)phenol)], phenylene-1,4-bis(2-benzimidazyl)-3,3′,5,5′-tetrasulphonic acid and/or its salts, 1,4-di(2-oxo-10-sulpho-3-bornylidenemethyl)benzene and/or salts thereof and/or 2,4-bis-{[4-(2-ethyl-hexyloxy)-2-hydroxy]phenyl}-6-(4-methoxyphenyl)-1,3,5-triazine, in each case individually or in any combinations with one another.

Other UV filter substances which have the structural formula 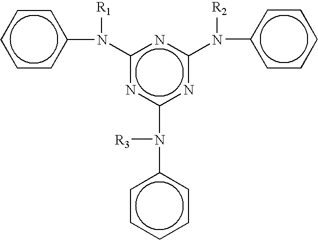
are also advantageous UV filter substances for the purposes of the present invention, for example the s-triazine derivatives which are described in the European Application EP 570 838 A1 and whose chemical structure is given by the generic formula 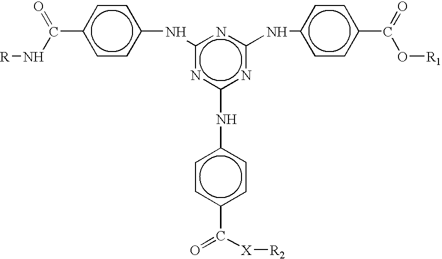
where
R is a branched or unbranched C₁-C₁₈-alkyl radical, a C₅-C₁₂-cycloalkyl radical, optionally substituted by one or more C₁-C₄-alkyl groups,
X is an oxygen atom or an NH group,
R₁ is a branched or unbranched C₁-C₁₈-alkyl radical, a C₅-C₁₂-cycloalkyl radical, optionally substituted by one or more C₁-C₄-alkyl groups, or a hydrogen atom, an alkali metal atom, an ammonium group or a group of the formula 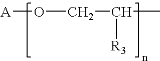
   in which
   A is a branched or unbranched C₁-C₁₈-alkyl radical, a C₅-C₁₂-cycloalkyl or aryl radical, optionally substituted by one or more C₁-C₄-alkyl groups,
   R₃ is a hydrogen atom or a methyl group,
   n is a number from 1 to 10,
R₂ is a branched or unbranched C₁-C₁₈-alkyl radical, a C₅-C₁₂-cycloalkyl radical, optionally substituted by one or more C₁-C₄-alkyl groups, if X is the NH group, and a branched or unbranched C₁-C₁₈-alkyl radical, a C₅-C₁₂-cycloalkyl radical, optionally substituted by one or more C₁-C₄-alkyl groups, or a hydrogen atom, an alkali metal atom, an ammonium group or a group of the formula 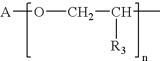
   in which
   A is a branched or unbranched C₁-C₁₈-alkyl radical, a C₅-C₁₂-cycloalkyl or aryl radical, optionally substituted by one or more C₁-C₄-alkyl groups,
   R₃ is a hydrogen atom or a methyl group,
   n is a number from 1 to 10,
   if X is an oxygen atom.

For the purposes of the present invention, a particularly preferred UV filter substance is also an asymmetrically substituted s-triazine, the chemical structure of which is given by the formula 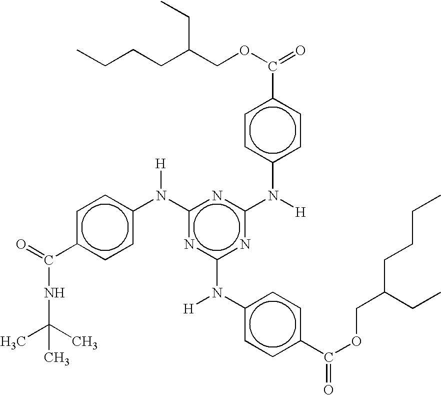
which is also referred to below as dioctylbutylamidotriazone (INCI: Dioctylbutamidotriazone) and is available under the trade name UVASORB HEB from Sigma 3V.

Also advantageous for the purposes of the present invention is a symmetrically substituted s-triazine, tris(2-ethylhexyl) 4,4′,4″-(1,3,5-triazine-2,4,6-triyltriimino)tris-benzoate, synonym: 2,4,6-tris[anilino-(p-carbo-2′-ethyl-1′-hexyloxy)]-1,3,5-triazine (INCI: Octyl Triazone), which is sold by BASF Aktiengesellschaft under the trade name UVINUL® T 150.

European Patent Application EP 775 698 also describes bisresorcinyltriazine which may preferably be used, the chemical structure of which is given by the formula 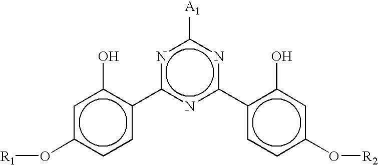
where R₁, R₂ and A₁ represent very diverse organic radicals. Also advantageous for the purposes of the present invention are 2,4-bis{[4-(3-sulphonato)-2-hydroxypropyloxy)-2-hydroxy]phenyl}-6-(4-methoxyphenyl)-1,3,5-triazine sodium salt, 2,4-bis{[4-(3-(2-propyloxy)-2-hydroxypropyloxy)-2-hydroxy]phenyl}-6-(4-methoxyphenyl)-1,3,5-triazine, 2,4-bis{[4-(2-ethylhexyloxy)-2-hydroxy]phenyl}-6-[4-(2-methoxyethylcarboxyl)phenylamino]-1,3,5-triazine, 2,4-bis{[4-(3-(2-propyloxy)-2-hydroxypropyloxy)-2-hydroxy]phenyl}-6-[4-(2-ethylcarboxyl)phenylamino]-1,3,5-triazine, 2,4-bis{[4-(2-ethylhexyloxy)-2-hydroxy]phenyl}-6-(1-methyl-pyrrol-2-yl)-1,3,5-triazine, 2,4-bis{[4-tris(trimethylsiloxysilylpropyloxy)-2-hydroxy]phenyl}-6-(4-methoxyphenyl)-1,3,5-triazine, 2,4-bis{[4-(2″-methylpropenyloxy)-2-hydroxy]phenyl}-6-(4-methoxyphenyl)-1,3,5-triazine and 2,4-bis{[4-(1′,1′,1′,3′,5′,5′,5′-heptamethylsiloxy-2″-methylpropyloxy)-2-hydroxy]phenyl}-6-(4-methoxyphenyl)-1,3,5-triazine.

An advantageous broadband filter for the purposes of the present invention is 2,2′-methylenebis(6-(2H-benzotriazol-2-yl)-4-(1,1,3,3-tetramethylbutyl)phenol), which is available under the trade name Tinosorb® M from CIBA-Chemikalien GmbH.

An advantageous broadband filter for the purposes of the present invention is also 2-(2H-benzotriazol-2-yl)-4-methyl-6-[2-methyl-3-[1,3,3,3-tetramethyl-1-[(trimethylsilyl)oxy]disiloxanyl]propyl]phenol (CAS-No.: 155633-54-8) with the INCI name Drometrizole Trisiloxane.

The UV-B and/or broadband filters may be oil-soluble or water-soluble. Advantageous oil-soluble UV-B and/or broadband filter substances are, for example:
3-benzylidenecamphor derivatives, preferably 3-(4-methylbenzylidene)camphor, 3-benzylidenecamphor;
4-aminobenzoic acid derivatives, preferably 2-ethylhexyl 4-(dimethylamino)benzoate, amyl 4-(dimethylamino)benzoate;
2,4,6-trianilino-(p-carbo-2′-ethyl-1′-hexyloxy)-1,3,5-triazine;
esters of benzalmalonic acid, preferably di(2-ethylhexyl) 4-methoxybenzalmalonate;
esters of cinnamic acid, preferably 2-ethylhexyl 4-methoxycinnamate, isopentyl 4-methoxycinnamate;
derivatives of benzophenone, preferably 2-hydroxy-4-methoxybenzophenone, 2-hydroxy-4-methoxy-4′-methylbenzophenone, 2,2′-dihydroxy-4-methoxybenzophenone
and UV filters bound to polymers.

Advantageous water-soluble UV-B and/or broadband filter substances are, for example:
salts of 2-phenylbenzimidazole-5-sulphonic acid, such as its sodium, potassium or its triethanolammonium salt, and the sulphonic acid itself;
sulphonic acid derivatives of 3-benzylidenecamphor, such as, for example, 4-(2-oxo-3-bornylidenemethyl)benzenesulphonic acid, 2-methyl-5-(2-oxo-3-bornylidenemethyl)sulphonic acid and salts thereof.

UV filter substances which are liquid at room temperature and are particularly advantageous for the purposes of the present invention are homomenthyl salicylate (INCI: Homosalate), 2-ethylhexyl 2-hydroxybenzoate (2-ethylhexyl salicylate, octyl salicylate, INCI: Octyl Salicylate), 4-isopropylbenzyl salicylate and esters of cinnamic acid, preferably 2-ethylhexyl 4-methoxycinnamate (INCI: Octyl Methoxycinnamate) and isopentyl 4-methoxycinnamate (INCI: Isoamyl p-Methoxycinnamate), 3-(4-(2,2-bisethoxycarbonylvinyl)phenoxy)propenyl)methoxysiloxane/dimethylsiloxane copolymer (INCI: Dimethicodiethyl Benzalmalonate), which is available, for example, under the trade name Parsol® SLX from Hoffmann La Roche.

A further photoprotective filter substance to be used advantageously according to the invention is ethylhexyl 2-cyano-3,3-diphenylacrylate (octocrylene), which is available from BASF under the name Uvinul® N 539.

A benzoxazole derivative which is particularly preferred according to the invention is 2,4-bis[5-(dimethylpropyl)benzoxazol-2-yl-(4-phenyl)imino]-6-(2-ethylhexyl)imino-1,3,5-triazine with the CAS No. 288254-16-0, which is characterized by the structural formula 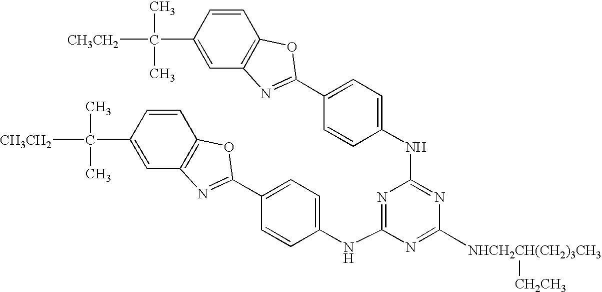
and is available from 3V Sigma under the trade name Uvasorb® K2A.

It may also be of considerable advantage to use polymer-bound or polymeric UV filter substances in preparations according to the present invention, in particular those as described in WO-A-92/20690.

The list of specified UV filters which can be used for the purposes of the present invention is not of course intended to be limiting.

Advantageously, the preparations according to the invention comprise the substances which absorb UV radiation in the UV-A and/or UV-B region, in a total amount of, for example, 0.1% by weight to 80% by weight, preferably 0.5 to 70% by weight, in particular 1.0 to 60% by weight, in each case based on the total weight of the preparations, in order to make available cosmetic preparations which protect the hair and/or the skin from the entire range of ultraviolet radiation. They can also serve as sunscreen compositions for the hair or the skin.

The weight data refer here to the composition of the preparation before drying by means of freeze-drying and/or spray-drying.

Particularly preferred embodiments of the present invention comprise, as UV filters, one or more triazine derivatives, dibenzoylmethane derivatives, UV filters liquid at room temperature and/or inorganic pigments, in particular titanium dioxide. In addition, it may in some cases be advantageous to incorporate film formers into the emulsion according to the invention, for example in order to improve the water resistance of the preparations or to increase the UV protection performance (UV-A and/or UV-B boosting). Both water-soluble or dispersible and also fat-soluble film formers are suitable, in each case individually or in combination with one another.

Advantageous water-soluble or dispersible film formers are, for example, polyurethanes (e.g. the Avalure® grades from Goodrich), dimethicone copolyol polyacrylates (Silsoft Surface® from Witco Organo Silicones Group), PVP/VA (VA=vinyl acetate) copolymer (Luviscol VA 64 Powder from BASF) etc.

Advantageous water-soluble film formers are, for example, the film formers from the group of polymers based on polyvinylpyrrolidone (PVP)

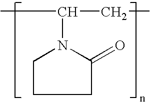

Particular preference is given to copolymers of polyvinylpyrrolidone, for example the PVP hexadecene copolymer and the PVP eicosene copolymer, which are available under the trade names Antaron V216 and Antaron V220 from GAF Chemicals Cooperation, and Tricontayl PVP and the like.

According to the invention, the customary antioxidants can be used in the emulsion. The antioxidants are advantageously chosen from amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. α-carotene, β-carotene, lycopene) and derivatives thereof, aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and (metal) chelators (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, alaninediacectic acid, flavonoids, polyphenols, catechins, vitamin C and derivatives (e.g. ascorbyl palmitate, Mg ascorbyl phosphate, ascorbyl acetate), tocopherols and derivatives (e.g. vitamin E acetate), and coniferyl benzoate of benzoin resin, rutinic acid and derivatives thereof, ferulic acid and derivatives thereof, butylhydroxytoluene, butylhydroxyanisole, nordihydroguaiac resin acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives suitable according to the invention (salts, esters, ethers, sugars, nucleotides, nucleosides, peptides and lipids) of these specified active ingredients.

The amount of the antioxidants (one or more compounds) in the preparations is preferably 0.001 to 30% by weight, particularly preferably 0.025-6.0% by weight, in particular 0.05-3.0% by weight, based on the total weight of the preparation. The weight data refer here to the composition of the preparation before drying by mans of freeze-drying and/or spray-drying.

If vitamin A or vitamin A derivatives, or carotenes or derivatives thereof are the antioxidant or the antioxidants, it is advantageous to choose their respective concentrations from the range of from 0.001 to 30% by weight, based on the total weight of the formulation. The weight data here refer to the composition of the preparation before drying by means of freeze-drying and/or spray-drying.

If vitamin E and/or derivatives thereof are the antioxidant or the antioxidants, it is advantageous to choose their respective concentrations from the range from 0.001 to 30% by weight, based on the total weight of the formulation. The weight data here refer to the composition of the preparation before drying by means of freeze-drying and/or spray-drying. Further advantageous active ingredients for the purposes of the present invention are natural active ingredients and/or derivatives thereof, such as, for example, alpha-lipoic acid, phytoene, D-biotin, coenzyme Q10, alpha-glucosylrutin, carnitine, carnosine, natural and/or synthetic isoflavonoids, creatin, fumaric esters, ectoin and derivatives thereof, taurine, and/or β-alanine. Based on the total weight of the emulsion, these active ingredients may be present therein in a concentration of from 0.001 to 30% by weight. The weight data here refer to the composition of the preparation before drying by means of freeze-drying and/or spray-drying.

Formulations according to the invention which comprise, for example, known antiwrinkle active ingredients, such as flavone glycosides (in particular α-glycosylrutin), coenzyme Q10, vitamin E and/or derivatives and the like are particularly advantageously suitable for the prophylaxis and treatment of cosmetic or dermatological changes in the skin, as arise, for example, during skin ageing (such as, for example, dryness, roughness and formation of dryness wrinkles, itching, reduced regreasing (e.g. after washing), visible vascular dilations (teleangiectases, couperosis), flaccidity and formation of lines and wrinkles, local hyperpigmentation, hypopigmentation and incorrect pigmentation (e.g. age spots), increased susceptibility to mechanical stress (e.g. cracking) and the like). In addition, they are advantageously suitable for combating the appearance of dry and/or rough skin.

However, other pharmaceutically or dermatologically effective substances, such as, for example, substances which calm and care for the skin, can also be incorporated into the preparations according to the invention. These include, for example, panthenol, allantoin, tannin, antihistamines (e.g. loratadine, cetirizine, dimethindene, clemastine, capsaicin, H₁ antagonists, tannin preparations), local anaesthetics, opiate antagonists (e.g. naltrexone, naloxone), antiphlogistics, glucocorticoids (e.g. hydrocortisone, tacrolimus, ciclosporin A) and plant active ingredients, such as azulene and bisabolol, glycyrrhizin, hamamelin and plant extracts, such as camomile, aloe vera, hamamelis, liquorice. The vitamin D₃ analogues tacalcitol, calcipotriol, colecalciferol and calcitrol (vitamin D₃) and/or fumaric esters can also be successfully incorporated into the preparations. Based on the total weight of the emulsion, these active ingredients may be present therein in a concentration of from 0.001 to 30% by weight. The weight data here refer to the composition of the preparation before drying by means of freeze-drying and/or spray-drying.

Advantageous moisturizing and humectant agents (so-called moisturizers) for the purposes of the present invention are, for example, sorbitol, mannitol, glycerol, lactic acid and/or lactates, in particular sodium lactate, butylene glycol, propylene glycol, biosaccaride gum-1, glycine soya, ethylhexyloxyglycerol, pyrrolidonecarboxylic acid and urea. In addition, it is particularly advantageous to use polymeric moisturizers from the group of water-soluble and/or water-swellable and/or water-gellable polysaccharides. Of particular advantage are, for example, hyaluronic acid, chitosan and/or a fucose-rich polysaccharide, which is deposited in Chemical Abstracts under the registry number 178463-23-5 and is available, for example, under the name Fucogel®1000 from SOLABIA S.A.

The cosmetic or dermatological preparations according to the invention may also advantageously, but not necessarily, comprise fillers which, for example, further improve the sensory and cosmetic properties of the formulation and bring about or enhance a velvety or silky feel on the skin. Advantageous fillers for the purposes of the present invention are starch and starch derivatives (such as, for example, tapioca starch, distarch phosphate, aluminium or sodium starch octenylsuccinate and the like), pigments which have neither primarily a UV filter effect nor colouring effect (such as, for example, boron nitride etc.) and/or Aerosils® (CAS No. 7631-86-9).

According to the invention, it is also advantageous to add hydroxypropylmethylcellulose to the emulsion according to the invention.

Apart from the above-mentioned substances, according to the invention, the compositions may optionally comprise additives customary in cosmetics, for example perfume, dyes, antimicrobial substances, regreasing agents, complexing and sequestering agents, pearlescent agents, plant extracts, vitamins, active ingredients, preservatives, bactericides, repellents, self-tanning agents (e.g. DHA), depigmenting agents (e.g. 8-hexadecene-1,16-dicarboxylic acid (dioic acid, CAS Number 20701-68-2; provisional INCI name Octadecenedioic acid)), pigments which have a colouring effect, softening, moisturizing and/or humectant substances, O/W emulsifiers, W/O emulsifiers, S/W emulsifiers, W/S emulsifiers or other customary constituents of a cosmetic or dermatological formulation, such as polymers, foam stabilizers, peeling substances (abrasives, e.g. polymer beads or powders made of polyethylene, polypropylene etc. inorganic oxides, silicates etc.), antiperspirant salts (e.g. acidic aluminium and/or aluminium/zirconium salts, such as aluminium chlorohydrate and/or aluminium/zirconium chlorohydrate) and electrolytes.

According to the invention, the repellent active ingredients listed below are preferred:

| | | | Effectiveness |
|---|---|---|---|
| | | | (literature and |
| | | | manufacturer |
| Chemical name | Trade name | Structure | details) |
| Butopyronoxyl | Indalone | 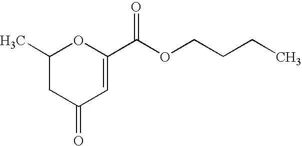 | biting insects¹ |
| 2,3;4,5-bis(2- butylene)tetra- hydro-2- furaldehyde | MGK- Repellent 11 | 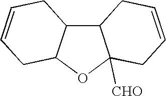 | cockroaches and biting insects¹ |
| N,N-diethylcaprylamide | Repellent 790 | 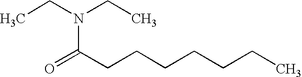 | cockroaches, mosquitoes, houseflies, horseflies, ants, arachnids, |
| o-chloro-N,N-di- ethylbenzamide in a mixture with N,N-diethylbenz- amide | Kik- Repellent | 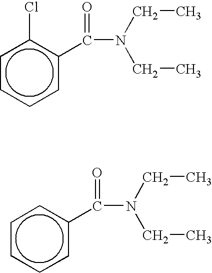 | mosquitoes, horseflies, fleas bugs, ticks, flies, lice |
| dimethyl carbate | Dimalone | 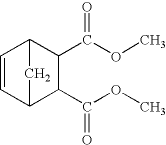 | mosquitoes, in particular of the Aedes species¹ |
| di-n-propyl iso- cinchomeronate | MGK- Repellent 326 | 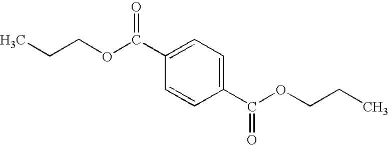 | houseflies, bush flies¹ |
| 2-ethylhexane- 1,3-diol | Rutgers 612 | 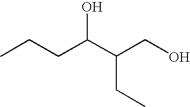 | mosquitoes, horseflies, flies, fleas, mites¹ |
| N-octylbicyclo- heptenedicarboxi- mide | MGK 264 Insecticide- synergist | 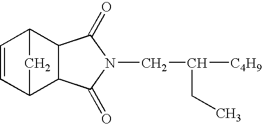 | Synergist² |
| Piperonyl butoxide | PBO | 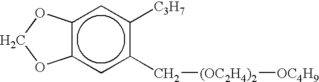 | Synergist² |

¹predominantly in a mixture or combination with other repellents
²acts as synergist with various repellents

Particularly advantageous repellent active ingredients for the purposes of the present invention are the above-mentioned active ingredients N,N-diethyl-3-methylbenzamide, ethyl 3-(N-n-butyl-N-acetylamino)propionate and dimethyl phthalate. Very particular preference is given to the repellent ethyl 3-(N-n-butyl-N-acetylamino)propionate.

Embodiments of the emulsion according to the invention which are advantageous according to the invention comprise one or more repellent active ingredients in a concentration of 1-50% by weight, based on the total weight of the formulation. The weight data here refer to the composition of the preparation before drying by means of freeze-drying and/or spray-drying.

Self-tanning agents which can be used advantageously according to the invention are, inter alia: glycerol aldehyde, hydroxymethylglyoxal, γ-dialdehyde, erythrulose, 6-aldo-D-fructose, ninhydrin, 5-hydroxy-1,4-naphthoquinone (Juglon), 2-hydroxy-1,4-naphthoquinone (Lawson) and particularly preferably 1,3-dihydroxyacetone.

Embodiments advantageous according to the invention with at least one self-tanning substance comprise these in a total concentration of from 0.1 to 30% by weight, based on the total weight of the emulsion. The weight data here refer to the composition of the preparation before drying by means of freeze-drying and/or spray-drying.

According to the invention, the emulsion according to the invention can advantageously comprise one or more preservatives. Advantageous preservatives for the purposes of the present invention are, for example, formaldehyde donors (such as, for example, DMDM hydantoin, which is available, for example, under the trade names Glydant™ from Lonza), iodopropynyl butylcarbamates (e.g. both available under the trade names Glycacil-L, Glycacil-S from Lonza and/or Dekaben LMB from Jan Dekker), parabens (i.e. alkyl p-hydroxybenzoates, such as methyl, ethyl, propyl and/or butyl paraben), phenoxyethanol, ethanol, benzoic acid and the like. In addition, according to the invention, the preservatives system usually also advantageously comprises preserving aids, such as, for example, octoxyglycerol, glycine soya etc. The table below gives an overview of some preservatives which are advantageous according to the invention:

| | |
|---|---|
| E 200 | sorbic acid |
| E 201 | sodium sorbate |
| E 202 | potassium sorbate |
| E 203 | calcium sorbate |
| E 210 | benzoic acid |
| E 211 | sodium benzoate |
| E 212 | potassium benzoate |
| E 213 | calcium benzoate |
| E 214 | ethyl p-hydroxybenzoate |
| E 215 | ethyl p-hydroxybenzoate Na salt |
| E 216 | n-propyl p-hydroxybenzoate |
| E 217 | n-propyl p-hydroxybenzoate Na salt |
| E 218 | methyl p-hydroxybenzoate |
| E 219 | methyl p-hydroxybenzoate Na salt |
| E 220 | sulphur dioxide |
| E 221 | sodium sulphite |
| E 222 | sodium hydrogensulphite |
| E 223 | sodium disulphite |
| E 224 | potassium disulphite |
| E 226 | calcium sulphite |
| E 227 | calcium hydrogensulphite |
| E 228 | potassium hydrogensulphite |
| E 230 | biphenyl (diphenyl) |
| E 231 | orthophenylphenol |
| E 232 | sodium orthophenylphenoxide |
| E 233 | thiabendazole |
| E 235 | natamycin |
| E 236 | formic acid |
| E 237 | sodium formate |
| E 238 | calcium formate |
| E 239 | hexamethylenetetramine |
| E 249 | potassium nitrite |
| E 250 | sodium nitrite |
| E 251 | sodium nitrate |
| E 252 | potassium nitrate |
| E 280 | propionic acid |
| E 281 | sodium propionate |
| E 282 | calcium propionate |
| E 283 | potassium propionate |
| E 290 | carbon dioxide |

Also advantageous are preservatives or preserving auxiliaries customary in cosmetics, such as dibromodicyanobutane (2-bromo-2-bromomethylglutaronitrile), phenoxyethanol, 3-iodo-2-propynyl butylcarbamate, 2-bromo-2-nitropropane-1,3-diol, imidazolidinylurea, 5-chloro-2-methyl-4-isothiazolin-3-one, 2-chloroacetamide, benzalkonium chloride, benzyl alcohol, salicylic acid and salicylates. According to the invention, it is particularly preferred if the preservatives used are iodopropynyl butylcarbamates, parabens (methyl, ethyl, propyl and/or butyl paraben) and/or phenoxyethanol.

According to the invention, one or more preservatives are advantageously present in a concentration of 2% by weight or less than 2% by weight, preferably 1.5% by weight or less than 1.5% by weight and particularly preferably 1% by weight or less than 1% by weight, in each case based on the total weight of the preparation. The weight data here refer to the composition of the preparation before drying by means of freeze-drying and/or spray-drying.

The emulsion according to the invention advantageously comprises one or more conditioners. Conditioners preferred according to the invention are, for example, all compounds which are listed in the International Cosmetic Ingredient Dictionary and Handbook (Volume 4, editors: R. C. Pepe, J. A. Wenninger, G. N. McEwen, The Cosmetic, Toiletry, and Fragrance Association, 9th edition, 2002) under Section 4 under the keywords Hair Conditioning Agents, Humectants, Skin-Conditioning Agents, Skin-Conditioning AgentsEmollient, Skin-Conditioning Agents-Humectant, Skin-Conditioning Agents-Miscellaneous, Skin-Conditioning Agents-Occlusive and Skin Protectants, and all of the compounds listed in EP 0934956 (pp. 11-13) under water soluble conditioning agent and oil soluble conditioning agent. Some of these compounds are listed by name under the constituents of the aqueous phase and of the oil phase. Further conditioners advantageous according to the invention are, for example, the compounds named Polyquaternium according to the international nomenclature for cosmetic ingredients (INCI) (in particular Polyquaternium-1 to Polyquaternium-56).

According to the invention, the emulsion according to the invention can advantageously comprise glitter substances and/or other effect substances.

For the purposes of the present invention, it is advantageous if the emulsion according to the invention comprises one or more hydrocolloids in a concentration of from 0.01 to 40% by weight, preferably in a concentration of from 0.1 to 35% by weight and very particularly preferably in a concentration of from 0.3 to 30% by weight, in each case based on the total weight of the preparation. The weight data here refer to the composition of the preparation before drying by means of freeze-drying and/or spray-drying.

Hydrocolloids advantageous according to the invention which are used are agar agar, carrageen, tragacanth, gum arabic, alginates, pectins, polyoses, guar flour, carob seed flour, starch, dextrins, polysaccharide N-alkylurethanes, inulin carbamates, gelatine, casein, cellulose ethers, hydroxyethyl- and -propylcellulose derivatives, polysaccharides, polyacrylic and polymethacrylic compounds, ammonium acryloyldimethyltaurates/vinylpyrrolidone-copolymers and ammonium polyacryloyldimethyltauramides, vinyl polymers, polycarboxylic acids, polyethers, polyimines, polyamides, polysilicic acids, clay minerals, zeolites, silicas.

Hydrocolloids preferred according to the invention are, for example, methylcelluloses and hydroxyethylcelluloses, which are the names for the methyl ether and the hydroxyethyl ester, respectively, of cellulose. They are characterized by the following structural formula 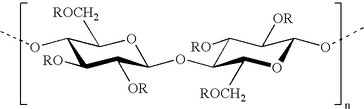
in which R can be a hydrogen, a methyl group or a hydroxyethyl group.

For the purposes of the present invention, the cellulose mixed ethers, which are generally likewise referred to as methylcelluloses and which, besides a dominant content of methyl groups, additionally comprise 2-hydroxyethyl, 2-hydroxypropyl or 2-hydroxybutyl groups, are particularly advantageous. Particular preference is given to (hydroxypropyl)methylcelluloses, for example those available under the trade name Methocel E4M from Dow Chemical Comp.

Also advantageous according to the invention is sodium carboxymethylcellulose, the sodium salt of the glycolic acid ether of cellulose, for which R in the above structural formula can represent a hydrogen and/or CH₂—COONa. Particular preference is given to the sodium carboxymethylcellulose available under the trade name Natrosol Plus 330 CS from Aqualon, also referred to a cellulose gum.

For the purposes of the present invention, preference is also given to xanthan (CAS No. 11138-66-2), also called xanthan gum, which is an anionic heteropolysaccharide which is usually formed from corn sugar by fermentation and is isolated as the potassium salt.

An advantageous gel former for the purposes of the present invention is also carrageen, a gel-forming extract constructed like agar from North Atlantic red algae belonging to the Florideae (Chondrus crispus and Gigartina stellata).

Polyacrylates are gelling agents likewise to be used advantageously for the purposes of the present invention. Polyacrylates advantageous according to the invention are acrylate-alkyl acrylate copolymers, in particular those which are chosen from the so-called carbomers or carbopols (Carbopol® is actually a registered trademark of NOVEON Inc.). In particular, the acrylate-alkyl acrylate copolymers advantageous according to the invention are characterized by the following structure:

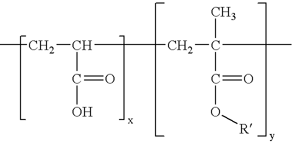

In this structure, R′ is a long-chain alkyl radical and x and y are numbers which symbolize the respective stoichiometric fraction of the particular comonomers.

According to the invention, particular preference is given to acrylate copolymers and/or acrylate-alkyl acrylate copolymers which are available under the trade names Carbopol® 1382, Carbopol® 981 and Carbopol® 5984, Aqua SF-1 from NOVEON Inc. and as Aculyn® 33 from International Specialty Products Corp. Further preference is given to the carbomers Carbopol EDT 2001, ETD 2020 and ETD 2050.

Also advantageous are copolymers of C10-30-alkyl acrylates and one or more monomers of acrylic acid, of methacrylic acid or esters thereof which are crosslinked with an allyl ether of sucrose or an allyl ether of pentaerythritol.

Compounds which bear the INCI name “Acrylates/C 10-30 Alkyl Acrylate Crosspolymer” are advantageous. Those available under the trade names Pemulen TR1 and Pemulen TR2 from NOVEON Inc. are particularly advantageous.

Compounds which bear the INCI name “Acrylates/Vinyl Isodecanoate Crosspolymer” are advantageous. Those available under the trade name Stabylen 30 from 3V Sigma are particularly advantageous.

Also advantageous are compounds which have the INCI name “acrylates/C12-24 pareth-25 acrylate copolymer” (available under the trade names Synthalens W2000 from 3V Inc.), which have the INCI name “acrylates/steareth-20 methacrylate copolymer” (available under the trade names Aculyn® 22 from the International Specialty Products Corp.), which have the INCI name “acrylates/steareth-20 itaconate copolymer” (available under the trade names Structure 2001® from National Starch), which have the INCI name “acrylates/aminoacrylates/C₁₀-30 alkyl PEG-20 itaconate copolymer” (available under the trade names Structure Plus® from National Starch) and similar polymers.

According to the invention, it is particularly preferred to use neutralized or partially neutralized polyacrylates (e.g. Carbopols from Noveon).

The cosmetic and/or dermatological emulsions according to the invention can comprise a number of pigments. The dyes and coloring pigments can be chosen from the corresponding positive list of cosmetics legislation or the EC list of cosmetic colorants. In most cases, they are identical to the dyes approved for foods. Advantageous color pigments are, for example, titanium dioxide, mica, iron oxides (e.g. Fe₂O₃, Fe₃O₄, FeO(OH)) and/or tin oxide. Advantageous dyes are, for example, carmine, Prussian blue, chromium oxide green, ultramarine blue and/or manganese violet. It is particularly advantageous to choose the dyes and/or colour pigments from the following list. The Colour Index numbers (CIN) can be found in the Rowe Colour Index, 3rd edition, Society of Dyers and Colourists, Bradford, England, 1971.

| Chemical or other name | CIN | Colour |
|---|---|---|
| Pigment Green | 10006 | green |
| Acid Green 1 | 10020 | green |
| 2,4-dinitrohydroxynaphthalene-7-sulphonic acid | 10316 | yellow |
| Pigment Yellow 1 | 11680 | yellow |
| Pigment Yellow 3 | 11710 | yellow |
| Pigment Orange 1 | 11725 | orange |
| 2,4-dihydroxyazobenzene | 11920 | orange |
| Solvent Red 3 | 12010 | red |
| 1-(2′-chloro-4′-nitro-1′-phenylazo)-2-hydroxynaphthalene | 12085 | red |
| Pigment Red 3 | 12120 | red |
| Ceres Red; Sudan Red; Fat Red G | 12150 | red |
| Pigment Red 112 | 12370 | red |
| Pigment Red 7 | 12420 | red |
| Pigment Brown 1 | 12480 | brown |
| 4-(2′-methoxy-5′-sulphonic acid diethylamide-1′-phenylazo)-3- | 12490 | red |
| hydroxy-5″-chloro-2″,4″-dimethoxy-2-naphthoic acid anilide | | |
| Disperse Yellow 16 | 12700 | yellow |
| 1-(4-sulpho-1-phenylazo)-4-aminobenzene-5-sulphonic acid | 13015 | yellow |
| 2,4-dihydroxyazobenzene-4′-sulphonic acid | 14270 | orange |
| 2-(2,4-dimethylphenylazo-5-sulphonic acid)-1- | 14700 | red |
| hydroxynaphthalene-4-sulphonic acid | | |
| 2-(4-sulpho-1-naphthylazo)-1-naphthol-4-sulphonic acid | 14720 | red |
| 2-(6-sulpho-2,4-xylylazo)-1-naphthol-5-sulphonic acid | 14815 | red |
| 1-(4′-sulphophenylazo)-2-hydroxynaphthalene | 15510 | orange |
| 1-(2-sulphonic acid-4-chloro-5-carboxylic acid-1-phenylazo)-2- | 15525 | red |
| hydroxynaphthalene | | |
| 1-(3-methylphenylazo-4-sulphonic acid)-2- | 15580 | red |
| hydroxynaphthalene | | |
| 1-(4′,(8′)-sulphonic acid-naphthylazo)-2-hydroxynaphthalene | 15620 | red |
| 2-hydroxy-1,2′-azonaphthalene-1′-sulphonic acid | 15630 | red |
| 3-hydroxy-4-phenylazo-2-naphthylcarboxylic acid | 15800 | red |
| 1-(2-sulpho-4-methyl-1-phenylazo)-2-naphthylcarboxylic acid | 15850 | red |
| 1-(2-sulpho-4-methyl-5-chloro-1-phenylazo)-2-hydroxy- | 15865 | red |
| naphthalene-3-carboxylic acid | | |
| 1-(2-sulpho-1-naphthylazo)-2-hydroxynaphthalene-3- | 15880 | red |
| carboxylic acid | | |
| 1-(3-sulpho-1-phenylazo)-2-naphthol-6-sulphonic acid | 15980 | orange |
| 1-(4-sulpho-1-phenylazo)-2-naphthol-6-sulphonic acid | 15985 | yellow |
| Allura Red | 16035 | red |
| 1-(4-sulpho-1-naphthylazo)-2-naphthol-3,6-disulphonic acid | 16185 | red |
| Acid Orange 10 | 16230 | orange |
| 1-(4-sulpho-1-naphthylazo)-2-naphthol-6,8-disulphonic acid | 16255 | red |
| 1-(4-sulpho-1-naphthylazo)-2-naphthol-3,6,8-trisulphonic acid | 16290 | red |
| 8-amino-2-phenylazo-1-naphthol-3,6-disulphonic acid | 17200 | red |
| Acid Red 1 | 18050 | red |
| Acid Red 155 | 18130 | red |
| Acid Yellow 121 | 18690 | yellow |
| Acid Red 180 | 18736 | red |
| Acid Yellow 11 | 18820 | yellow |
| Acid Yellow 17 | 18965 | yellow |
| 4-(4-sulpho-1-phenylazo)-1-(4-sulphophenyl)-5-hydroxy- | 19140 | yellow |
| pyrazolone-3-carboxylic acid | | |
| Pigment Yellow 16 | 20040 | yellow |
| 2,6-(4′-sulpho-2″,4″-dimethyl)bis-phenylazo)1,3-dihydroxy-benzene | 20170 | orange |
| Acid Black 1 | 20470 | black |
| Pigment Yellow 13 | 21100 | yellow |
| Pigment Yellow 83 | 21108 | yellow |
| Solvent Yellow | 21230 | yellow |
| Acid Red 163 | 24790 | red |
| Acid Red 73 | 27290 | red |
| 2-[4′-(4″-sulpho-1″-phenylazo)-7′-sulpho-1′-naphthylazo]-1- | 27755 | black |
| hydroxy-7-aminonaphthalene-3,6-disulphonic acid | | |
| 4′-[(4″-sulpho-1″-phenylazo)-7′-sulpho-1′-naphthylazo]-1- | 28440 | black |
| hydroxy-8-acetylaminonaphthalene-3,5-disulphonic acid | | |
| Direct Orange 34, 39, 44, 46, 60 | 40215 | orange |
| Food Yellow | 40800 | orange |
| trans-β-apo-8′-carotenealdehyde (C₃₀) | 40820 | orange |
| trans-apo-8′-carotenic acid (C₃₀) ethyl ester | 40825 | orange |
| Canthaxanthin | 40850 | orange |
| Acid Blue 1 | 42045 | blue |
| 2,4-disulpho-5-hydroxy-4′-4″-bis(diethylamino)triphenyl-carbinol | 42051 | blue |
| 4-[(4-N-ethyl-p-sulphobenzylamino)phenyl-(4-hydroxy-2- | 42053 | green |
| sulphophenyl)(methylene)-1-(N-ethyl-N-p-sulphobenzyl)-2,5- | | |
| cyclohexadieneimine] | | |
| Acid Blue 7 | 42080 | blue |
| (N-ethyl-p-sulphobenzylamino)phenyl-(2-sulphophenyl)- | 42090 | blue |
| methylene(N-ethyl-N-p-sulphobenzyl)Δ^{2,5}- | | |
| cyclohexadieneimine | | |
| Acid Green 9 | 42100 | green |
| diethyldisulphobenzyldi-4-amino-2-chlorodi-2-methyl- | 42170 | green |
| fuchsonimmonium | | |
| Basic Violet 14 | 42510 | violet |
| Basic Violet 2 | 42520 | violet |
| 2′-methyl-4′-(N-ethyl-N-m-sulphobenzyl)amino-4″-(N-diethyl)- | 42735 | blue |
| amino-2-methyl-N-ethyl-N-m-sulphobenzylfuchsonimmonium | | |
| 4′-(N-dimethyl)amino-4″-(N-phenyl)aminonaphtho-N-dimethyl- | 44045 | blue |
| fuchsonimmonium | | |
| 2-hydroxy-3,6-disulpho-4,4′-bisdimethylamino- | 44090 | green |
| naphthofuchsonimmonium | | |
| Acid Red 52 | 45100 | red |
| 3-(2′-methylphenylamino)-6-(2′-methyl-4′-sulphophenylamino)- | 45190 | violet |
| 9-(2″-carboxyphenyl)xanthenium salt | | |
| Acid Red 50 | 45220 | red |
| phenyl-2-oxyfluorone-2-carboxylic acid | 45350 | yellow |
| 4,5-dibromofluorescein | 45370 | orange |
| 2,4,5,7-tetrabromofluorescein | 45380 | red |
| Solvent Dye | 45396 | orange |
| Acid Red 98 | 45405 | red |
| 3′,4′,5′,6′-tetrachloro-2,4,5,7-tetrabromofluorescein | 45410 | red |
| 4,5-diiodofluorescein | 45425 | red |
| 2,4,5,7-tetraiodofluorescein | 45430 | red |
| quinophthalone | 47000 | yellow |
| quinophthalonedisulphonic acid | 47005 | yellow |

It may also be favorable to choose as the dye one or more substances from the following group: 2,4-dihydroxyazobenzene, 1-(2′-chloro-4′-nitro-1′-phenylazo)-2-hydroxynaphthalene, Ceres Red, 2-(4-sulpho-1-naphthylazo)-1-naphthol-4-sulphonic acid, calcium salt of 2-hydroxy-1,2′-azonaphthalene-1′-sulphonic acid, calcium and barium salts of 1-(2-sulpho-4-methyl-1-phenylazo)-2-naphthylcarboxylic acid, calcium salt of 1-(2-sulpho-1-naphthylazo)-2-hydroxynaphthalene-3-carboxylic acid, aluminium salt of 1-(4-sulpho-1-phenylazo)-2-naphthol-6-sulphonic acid, aluminium salt of 1-(4-sulpho-1-naphthylazo)-2-naphthol-3,6-disulphonic acid, 1-(4-sulpho-1-naphthylazo)-2-naphthol-6,8-disulphonic acid, aluminium salt of 8-amino-2-phenylazo-1-naphthol-3,6-disulphonic acid, aluminium salt of 4-(4-sulpho-1-phenylazo)-1-(4-sulphophenyl)-5-hydroxy-pyrazolone-3-carboxylic acid, 4′-[(4″-sulpho-1″-phenylazo)-7′-sulpho-1′-naphthylazo]-1-hydroxy-8-acetylaminonaphthalene-3,5-disulphonic acid, aluminium and zirconium salts of 4,5-dibromofluorescein, aluminium and zirconium salts of 2,4,5,7-tetrabromo-fluoroscein, 3′,4′,5′,6′-tetrachloro-2,4,5,7-tetrabromofluorescein and its aluminium salt, aluminium salt of 2,4,5,7-tetraiodofluorescein, aluminium salt of quinophthalone-disulphonic acid, aluminium salt of indigodisulphonic acid, 4,4′-dimethyl-6,6′-dichlorothioindigo, complex salt (Na, Al, Ca) of carminic acid, red and black iron oxide (CIN: 77 491 (red) and 77 499 (black)), iron oxide hydrate (CIN: 77 492), manganese ammonium diphosphate (CIN 77745), ultramarine (CIN 77007) and titanium dioxide.

Emulsions according to the invention can comprise titanium dioxide, which may be present either in the rutile or the anatase crystal modification and, for the purposes of the present invention, is advantageously surface-treated (“coated”), the intention being, for example, to form and/or retain a hydrophilic, amphiphilic or hydrophobic character. This surface treatment can consist in providing the pigments with a thin hydrophilic and/or hydrophobic inorganic and/or organic layer by processes known per se. The various surface coatings can also comprise water for the purposes of the present invention.

Inorganic surface coatings for the purposes of the present invention can consist of aluminium oxide (Al₂O₃), aluminium hydroxide Al(OH)₃, or aluminium oxide hydrate (also: alumina, CAS No.: 1333-84-2), sodium hexametaphosphate (NaPO₃)₆, sodium metaphosphate (NaPO₃)ₙ, silicon dioxide (SiO₂) (also: silica, CAS No.: 7631-86-9), zirconium oxide (ZrO₂) or iron oxide (Fe₂O₃). These inorganic surface coatings may be present on their own, in combination and/or in combination with organic coating materials. For this purpose, oxides, oxide hydrates or phosphates, for example of the elements Al, Si, Zr, are precipitated on the pigment surface in dense layers.

The inorganic after-treatment generally takes place in an aqueous suspension of the pigment by adding soluble after-treatment chemicals, such as, for example, aluminium sulphate, and subsequent precipitation of the hydroxide, which is sparingly soluble in the neutral range, by controlled adjustment of the pH with sodium hydroxide solution. After the inorganic after-treatment, the coated pigments are separated off from the suspension by filtration and washed carefully in order to remove the dissolved salts, and the isolated pigments are then dried.

Of particular preference for the purposes of this invention are titanium dioxides onto whose surface aluminium hydroxide has been applied, such as, for example, the titanium dioxide grades C47-051 and C47-5175 obtainable from Sun Chemical. Further preferred pigments are titanium dioxides which are coated with aluminium oxides and/or silicon oxides, such as, for example, from Krosnos Titan: Kronos 1071 and 1075 or from Kingfisher: A310.03 Tudor Aspen.

Organic surface coatings for the purposes of the present invention can consist of vegetable or animal aluminium stearate, vegetable or animal stearic acid, lauric acid, dimethylpolysiloxane (also: dimethicone), methylpolysiloxane (methicone), simethicone (a mixture of dimethylpolysiloxane with an average chain length of from 200 to 350 dimethylsiloxane units and silica gel) or alginic acid. These organic surface coatings may be present on their own, in combination and/or in combination with inorganic coating materials.

In addition, it may be advantageous according to the invention to use pearlescent pigments.

These include natural pearlescent pigments, such as, for example,
“fish silver” (guanine/hypoxanthine mixed crystals from fish scales) and
“mother-of-pearl” (ground mussel shells),
   monocrystalline pearlescent pigments, such as, for example, bismuth oxychloride (BiOCl),
   layer-substrate pigments: e.g. mica/metal oxide

The bases of pearlescent pigments are, for example, pulverulent pigments or castor oil dispersions of bismuth oxychloride and/or titanium dioxide, and bismuth oxychloride and/or titanium dioxide on mica. The lustre pigment listed under CIN 77163, for example, is particularly advantageous.

Also advantageous are, for example, the following pearlescent pigment types based on mica/metal oxide:

| | Coating/layer | |
|---|---|---|
| 1.1.1.1.1 Group | thickness | Colour |
| Silver-white pearlescent | TiO₂: 40-60 nm | silver |
| pigments | | |
| Interference pigments | TiO₂: 60-80 nm | yellow |
| | TiO₂: 80-100 nm | red |
| | TiO₂: 100-140 nm | blue |
| | TiO₂: 120-160 nm | green |
| Colour lustre pigments | Fe₂O₃ | bronze |
| | Fe₂O₃ | copper |
| | Fe₂O₃ | red |
| | Fe₂O₃ | red-violet |
| | Fe₂O₃ | red-green |
| | Fe₂O₃ | black |
| Combination pigments | TiO₂/Fe₂O₃ | golden shades |
| | TiO₂/Cr₂O₃ | green |
| | TiO₂/Prussian Blue | deep blue |
| | TiO₂/Carmine | red |

Particular preference is given to the pearlescent pigments obtainable from Merck under the trade names Timiron, Colorona or Dichrona.

The list of specified pearlescent pigments is not of course intended to be limiting. Pearlescent pigments advantageous for the purposes of the present invention are obtainable by numerous routes known per se. For example, substrates other than mica can be coated with further metal oxides, such as, for example, silica and the like. SiO₂ particles coated, for example, with TiO₂ and Fe₂O₃ (“Ronaspheres”), which are sold by Merck, are advantageous.

It may, moreover, be advantageous to completely dispense with a substrate such as mica. Particular preference is given to pearlescent pigments which are prepared using SiO₂. Such pigments, which can also additionally have goniochromatic effects, are obtainable, for example, under the trade name Sicopearl Fantastico from BASF.

Pigments from Engelhard/Mearl which are based on calcium sodium borosilicate and which are coated with titanium dioxide can also be used advantageously. These are available under the name Reflecks. Due to their particle size of 40-180 μm, they have a glitter effect in addition to the color.

The dyes and pigments can be present either individually or else in a mixture, and can also be mutually coated with one another, various color effects in general being brought about by different coating thicknesses. The total amount of the dyes and coloring pigments is advantageously chosen from the range from, for example, 0.1% by weight to 30% by weight, preferably from 0.5 to 15% by weight, in particular from 1.0 to 10% by weight, in each case based on the total weight of the preparations. The weight data here refer to the composition of the preparation before drying by means of freeze-drying and/or spray-drying.

For the purposes of the present invention, the O/W emulsion according to the invention advantageously comprises one or more tablet auxiliaries. These can be used according to the invention advantageously in a concentration of from 0.1 to 60% by weight, preferably in a concentration of from 1 to 50% by weight and very particularly preferably in a concentration of from 3 to 35% by weight, in each case based on the total weight of the preparation. The weight data here refer to the composition of the preparation, the preparation being regarded as being the emulsion after freeze-drying and/or spray-drying together with any further water-soluble and/or readily volatile compounds and tabletting substances (including bicarbonates and acids solid at room temperature). Tablet auxiliaries which may be used are, for example, filling and compacting auxiliaries (e.g. starch derivatives and/or cellulose derivatives), flow agents (e.g. highly disperse silicon dioxides), flow regulators, lubricants and mould release agents.

According to the invention, all substances can advantageously be used which are listed in H. P. Fielder, Lexikon der Hilfsstoffe für Pharmazie, Kosmetik und angrenzenden Gebiete [Lexicon of Auxiliaries for Pharmacy, Cosmetics and related fields], 5th edition, Editio Cantor Verlag, Aulendorf, 2002, under the keywords granulation auxiliaries, tablet binders, tablet fillers, tablet glidants, tablet auxiliaries, tablet disintegrants, tablet coatings.

In an embodiment which is particularly preferred according to the invention, the preparation according to the invention comprises a combination of carbonates and/or bicarbonates, and acids solid at room temperature (e.g. citric acid, ascorbic acid, lactic acid, tartaric acid, etc). Strongly swelling polymers, such as, for example, crosslinked polyvinylpyrrolidone, can also be used advantageously according to the invention.

According to the invention, one or more bicarbonates (e.g. sodium bicarbonate, potassium bicarbonate) are advantageously used in an amount of from 0.1 to 30% by weight, preferably in an amount of from 0.5 to 20% by weight and particularly preferably in an amount of from 0.5 to 15% by weight, in each case based on the total weight of the preparation, the preparation being regarded as being the emulsion after freeze-drying and/or spray-drying together with any further water-soluble and/or readily volatile compounds and tabletting substances (including bicarbonates and acids solid at room temperature).

According to the invention, one or more acids that are solid at room temperature (particularly preferably citric acid) are advantageously used in an amount of from 0.1 to 30% by weight, preferably in an amount of from 0.5 to 20% by weight and particularly preferably in an amount of from 0.5 to 15% by weight, in each case based on the total weight of the preparation, the preparation being regarded as being the emulsion after freeze-drying and/or spray-drying together with any further water-soluble and/or readily volatile compounds and tabletting substances (including bicarbonates and acids solid at room temperature).

Such an embodiment is especially advantageous for the purposes of the present invention if the emulsion according to the invention is in the form of a tablet (e.g. an effervescent tablet) since the emulsion according to the invention in this embodiment can be reemulsified particularly readily by adding water.

The invention also provides the process for preparing such a preparation, which is characterized in that the O/W emulsion according to the invention is mixed with one or more granulation auxiliaries in a mixing device and then granulated in a suitable granulation process.

The invention also provides the process for preparing such a preparation, which is characterized in that the O/W emulsion according to the invention is mixed with one or more tablet auxiliaries in a mixing device.

The invention also provides preparations which are prepared by these processes according to the invention.

The invention further provides a process for preparing tablets which is characterized in that a preparation according to the invention is pressed in a press to afford one or more tablets, and also preparations which are prepared by this process. Last but not least, the invention provides a process for preparing a cosmetic, which is characterized in that a preparation according to the invention (an O/w emulsion dried by spray-drying and/or freeze-drying which has optionally been admixed with water-soluble and/or readily volatile active ingredients, auxiliaries, additives and/or tablet auxiliaries, and has also optionally been tabletted and/or granulated) is admixed with water and/or emulsified in water. The invention also provides cosmetics which are prepared by this process.

Such a cosmetic according to the invention is then in the form of an ointment, cream, lotion or an emulsion foam (French: mousse), or in a sprayable form. According to the invention, it can be used advantageously for the treatment and care of the skin, hair and nails. According to the invention, preference is given here to the use as sunscreen composition.

### DETAILED DESCRIPTION OF THE INVENTION

The examples below serve to illustrate the present invention without limiting it. Table 1 describes exemplary compositions of the emulsion preparations prepared by the process according to the invention after spray-drying. Table 2 describes exemplary mixtures of substances which can be mixed homogeneously as the water-soluble/dispersible additives with the preparations prepared according to the invention.

Optionally, the mixtures of substances (Table 1+2) can be mixed with tabletting and granulation auxiliaries (Table 3) and granules and/or tablets according to the invention can be prepared by the process according to the invention.

### EXAMPLES

TABLE 1
Emulsion preparation after spray-/or freeze-drying

| | 1 | 2 | 3 | 4 | 5 |
|---|---|---|---|---|---|
| Titanium dioxide (Eusolex T2000) | 35 | 25 | 10 | | 45 |
| Zinc oxide | | 10 | 20 | 3 | |
| Titanium dioxide (Titanium dioxide T805) | 3 | | | 1 | |
| Silica (Aerosil R972) | 3 | 4 | | | 2 |
| Talc (Talc Micron) | 2 | | 1 | | |
| Boron nitride | | | 2 | | 0.5 |
| Sodium corn starch n-octenylsuccinate | | | | 2 | |
| Distarch phosphate | | 1.5 | 1 | | |
| Tapioca starch | 4 | | | 3 | |
| Polyamide-6 | | | | 4 | |
| Nylon-12 | | 3 | | | |
| C₂₀₋₄₀-Alkyl stearate (Kester wax K82) | | | 2 | | |
| Hydrogenated cocoglycerides | 3 | 1 | | 1 | |
| C₁₆₋₃₈-Alkylhydroxystearoyl stearate (Kester | | 0.5 | | | |
| wax K80P) | | | | | |
| Behenoxydimethicone (Abil Wax 2440) | | | 2 | | |
| Cetyldimethicone (Abil Wax 9840) | | 1.5 | | | |
| Polyisobutene (Rewopal PIB 1000) | | | 1.5 | | |
| Caprylic/capric triglyceride | | | 5 | 12 | |
| Octyldodecanol | 6 | 5 | 6 | 8 | |
| Mineral oil | 3.65 | | | 4 | 8 |
| Butylene glycol caprylate/caprate | 5 | 3 | | | |
| C₁₂₋₁₅-Alkyl benzoate | 6 | | 5 | | 5 |
| PVP/hexadecene copolymer | 0.5 | 0.5 | | | |
| Acetylated glycol stearate + tristearin | | | 3 | | |
| Stearyl alcohol | 1.5 | 1.5 | 1 | 1 | |
| Dimethicone | | | 2 | | |
| Cyclomethicone | 3 | 2 | | 3 | |
| Tocopheryl acetate | 1.5 | 1.5 | 2 | | |
| Ubiquinone | | | | 0.03 | |
| PEG-40 hydrogenated castor oil | 1.5 | | | | 2 |
| PPG-15 stearyl ether | | 3 | | | |
| Starch hydroxypropyltrimonium chloride | | | 1 | | |
| (Sensomer CI 50) | | | | | |
| Dicaprylyl ether (Cetiol OE) | | 3.5 | | | |
| Hydrogenated polyisobutene (Polysynlan) | | | | | |
| UVASorb ® K2A | 3 | | 4 | | |
| Butyl methoxydibenzoylmethane | | | | 3 | 5 |
| Uvinul ® A Plus | | 3 | | | |
| Bisethylhexyloxyphenol methoxyphenyltriazine | | 4 | | | 2 |
| Ethylhexyltriazone | 4 | | | 4 | 4.5 |
| Diethylhexylbutamidotriazone | | 5 | | | |
| Ethylhexyl methoxycinnamate | 5 | 5 | | 7 | 10 |
| Octocrylene | | 5.00 | 5 | 7.50 | |
| Methylenebisbenzotriazolyl | 3 | | 1.5 | | |
| tetramethylbutylphenol | | | | | |
| Homosalate | | 4 | | | |
| Ethylhexyl salicylate | | | 3 | | |
| Dihydroxyacetone | | | | 5 | |
| Diethylhexylbutamidotriazone | | 2 | | 2 | |
| (UVASORB HEB) | | | | | |
| Preservative | 2 | 0.5 | | 1 | 0.3 |
| Polysorbate 20 | | 2 | | | |
| Polysorbate 60 | | | 2 | | |
| Polysorbate 80 | | | | | 4 |
| Glyceryl stearate | | | | 3 | |
| Fat-soluble dye | | | | | 0.4 |
| Hydroxypropylmethylcellulose | | | 5 | 25 | |
| Hydroxyethylcellulose | | | | | 1 |
| Polyvinylpyrrolidone | 1 | | 10 | | 10 |
| Perfume | 1.5 | 1 | 0.5 | | 1 |
| Water | ad 100 | ad 100 | ad 100 | ad 100 | ad 100 |

TABLE 2
Water-soluble/-dispersible additives

| | 1 | 2 | 3 | 4 | 5 |
|---|---|---|---|---|---|
| Sodium corn starch n-octenylsuccinate | 2 | | | | 1 |
| Distarch phosphate | | | | 1.5 | |
| Tapioca starch | | 5 | | | 4 |
| Phenyldibenzimidazoletetrasulphonic acid | 2 | | | | |
| Phenylbenzimidazolesulphonic acid | 2 | | 2 | | |
| Preservative | | 0.5 | | 1 | |
| Glycerol | | | | | 5 |
| Panthenol | | 5 | | 1 | 0.1 |
| Sorbitol | 15 | | 7 | | |
| Mannitol | | 4 | | 10 | |
| Biosaccharide gel (Fucogel 1000) | | | 0.5 | | |
| Hyaluronic acid | | | 0.5 | | |
| Chitosan | 0.5 | | | | |
| Lactic acid | | | | 1 | |
| NaCl | | 1 | | | |
| Magnesium sulphate | 1 | | | | |
| Lactic acid | 0.6 | | | | |
| MgSO₄ | | 0.9 | | | |
| Carbomer (Carbopol 981) | 0.5 | | | 1 | |
| C₁₀₋₃₀-alkyl acrylate crosspolymer (Pemulen | | 0.3 | | | |
| TR1) | | | | | |
| Dimethicone/polysilicone-11 | | | 2 | | |
| Xanthan gum | | 0.1 | | | |
| Hydroxyethylcellulose | 1 | 0.5 | | | 2 |
| Hydroxypropylmethylcellulose | | 3 | | 2 | |
| Methylhydroxyethylcellulose | 0.1 | | | | |
| Polyquaternium 37 | | 0.5 | 1 | | |
| Polyvinylpyrrolidone | 2 | | | | 1 |
| Polyacrylate | | | | 0.5 | |
| NaOH 45% strength solution in water | 2 | | 0.7 | | |
| Water-soluble dye | | | 0.3 | | |
| Perfume | 0.5 | | 0.3 | | |

TABLE 3
Additives for granulating/tabletting the mixtures from 1 & 2

| | 1 | 2 | 3 | 4 | 5 |
|---|---|---|---|---|---|
| Microcrystalline cellulose | 15 | | | | 12 |
| (Avicel PH 102) | | | | | |
| Crosslinked sodium | 2 | | | 2 | 2 |
| carboxymethylcellulose | | | | | |
| (Ac-Di-Sol) | | | | | |
| Crosslinked polyvinylpyrrolidone | | | 5 | | 2 |
| Sodium hydrogencarbonate | | 2 | | 1 | |
| Citric acid | | 4.6 | | 2.3 | |
| Milk sugar | 3 | 5 | 4 | | 3 |
| Sucrose | | 6 | | 4 | |
| Gelatine | | | 1.5 | | |
| Starch | | 1 | | | |
| Glycerol | | 3 | | | |
| Sorbitol | 2 | | 3 | | 2 |
| Starch | | | | | |
| Aerosil 200 | | | 1.5 | 1 | |
| Stearic acid | 1.5 | | 1 | | 0.5 |
| Magnesium stearate | | 2 | | 1 | 1.5 |
| Talc | 1 | | 2 | | |
| Alcohol | | 20 | 15 | 10 | |

1. A process for preparing an O/W emulsion concentrate, which process comprises subjecting an O/W emulsion comprising
(a) 20% to 95% by weight of one or more lipids;
(b) 0.5% to 80% by weight of microfine solid particles selected from amphiphilic metal oxide particles, amphiphilic polymer particles, modified polysaccharide particles and combinations thereof; and
(c) water;
to at least one of spray-drying and freeze-drying to obtain an O/W emulsion concentrate.

2. The process of claim 1, wherein the O/W emulsion comprises at least 30% by weight of (a).

3. The process of claim 1, wherein the O/W emulsion comprises at least 50% by weight of (a).

4. The process of claim 2, wherein the O/W emulsion comprises from 1% to 50% by weight of (b).

5. The process of claim 3, wherein the O/W emulsion comprises at least 3% by weight of (b).

6. The process of claim 5, wherein the O/W emulsion comprises not more than 40% by weight of (b).

7. The process of claim 1, wherein the at least one of spray-drying and freeze-drying is conducted to a water content of less than 8% by weight.

8. The process of claim 4, wherein the at least one of spray-drying and freeze-drying is conducted to a water content of less than 5% by weight.

9. The process of claim 1, wherein the microfine solid particles have an average particle size of less than 100 μm.

10. The process of claim 4, wherein the microfine solid particles have an average particle size of less than 50 μm.

11. The process of claim 1, wherein (b) comprises metal oxide particles.

12. The process of claim 11, wherein the metal oxide comprises at least one of titanium dioxide, zinc oxide and iron oxides.

13. The process of claim 1, wherein the microfine solid particles comprise at least one of boron nitride, talc and latex.

14. The process of claim 1, wherein (b) comprises polymer particles.

15. The process of claim 14, wherein the polymer is selected from polycarbonates, polyethers, polyethylene, polypropylene, polyvinyl chloride, polystyrene, polyamides, polyacrylates and combinations thereof.

16. The process of claim 1, wherein (b) comprises modified polysaccharide particles.

17. The process of claim 16, wherein the modified polysaccharide comprises at least one of a starch ether and a starch ester.

18. The process of claim 17, wherein the modified polysaccharide particles have an average particle diameter of less than 20 μm.

19. The process of claim 1, wherein the O/W emulsion further comprises one or more polyols.

20. The process of claim 19, wherein the one or more polyols are present in a concentration of from 5% to 40% by weight.

21. A process for preparing an O/W emulsion concentrate, which process comprises subjecting an O/W emulsion comprising
(a) 50% to 95% by weight of one or more lipids;
(b) 3% to 40% by weight of microfine solid particles having an average particle size of less than 50 μm and being selected from amphiphilic particles of at least one of titanium dioxide, zinc oxide and iron oxides; amphiphilic particles of at least one of a polycarbonate, a polyether, polyethylene, polypropylene, polyvinyl chloride, polystyrene, a polyamide, and a polyacrylate; particles of at least one of a starch ether and a starch ester; and combinations thereof; and
(c) water;
to at least one of spray-drying and freeze-drying to obtain an O/W emulsion concentrate having a water content of less than 5% by weight.

22. The process of claim 1, further comprising mixing the O/W emulsion concentrate with one or more substances selected from water-soluble substances, readily volatile substances, auxiliaries and additives.

23. The process of claim 1, further comprising mixing the O/W emulsion concentrate with one or more substances selected from granulating agents and tablet auxiliaries to obtain a second mixture.

24. The process of claim 23, wherein the O/W emulsion concentrate is mixed with at least one substance selected from carbonates, bicarbonates and acids that are solid at room temperature.

25. The process of claim 23, which further comprises at least one of granulating the second mixture and pressing the second mixture into a tablet.

26. An O/W emulsion concentrate that is obtainable by the process of claim 1.

27. The O/W emulsion concentrate of claim 26, wherein the concentrate comprises less than 5% by weight of water.

28. A process for preparing an O/W emulsion, wherein the process comprises combining the O/W emulsion concentrate of claim 26 with water.

29. A mixture that is obtainable by the process of claim 22.

30. A mixture that is obtainable by the process of claim 23.

31. A pressed tablet that is obtainable by the process of claim 25.

32. A granule that is obtainable by the process of claim 25.

33. A cosmetic preparation which comprises the concentrate of claim 26.

34. The cosmetic preparation of claim 33, which further comprises one or more UV photoprotective filter substances.

35. A cosmetic preparation that is obtainable by the process of claim 28.

36. A composition for the preparation of an O/W emulsion, wherein the composition comprises a mixture of (a) one or more lipids, (b) microfine solid particles selected from amphiphilic metal oxide particles, amphiphilic polymer particles, modified polysaccharide particles and combinations thereof, and (c) at least one substance selected from carbonates, bicarbonates, and acids that are solid at room temperature.

37. The mixture of claim 36, wherein the at least one substance comprises one or more of sodium bicarbonate, potassium bicarbonate and citric acid.
